(19) United States
(12) Patent Application Publication
(10) Pub. No.: US 2011/0117626 A1
(43) Pub. Date: May 19, 2011

(54) Hydrophobic Interaction Chromatography Membranes, and Methods of Use Thereof

(76) Inventors: Elena N. Komkova, Hamilton (CA); Alicja M. Mika, Hamilton (CA); Marianne Pankratz, Caledonia (CA)

(21) Appl. No.: 12/946,053

(22) Filed: Nov. 15, 2010

Related U.S. Application Data
(60) Provisional application No. 61/261,009, filed on Nov. 13, 2009.

(51) Int. Cl. C12N 9/36; C07K 16/00; C07K 14/805; C07K 14/435; B32B 3/26; B32B 5/32; B32B 5/28; B32B 38/08

(52) U.S. Cl. 435/206; 530/387.1; 530/385; 530/350; 428/316.6; 428/315.7; 442/76; 156/77

(57) ABSTRACT

Described herein are composite materials and methods of using them for hydrophobic interaction chromatography (HIC). In certain embodiments, the composite material comprises a support member, comprising a plurality of pores extending through the support member; and a macroporous cross-linked gel, comprising a plurality of macropores, and a plurality of pendant hydrophobic moieties. In certain embodiments, the composite materials may be used in the separation or purification of a biological molecule or biological ion.

### RELATED APPLICATIONS

This application claims the benefit of priority to U.S. Provisional Patent Application Ser. No. 61/261,009, filed Nov. 13, 2009, the contents of which are hereby incorporated by reference in their entirety.

### BACKGROUND OF THE INVENTION

Hydrophobicity is generally defined as the repulsion between a non-polar compound and a polar environment, such as water. Hydrophobic “interactions” are essentially the physical manifestation of the tendency of a polar environment to exclude hydrophobic (i.e., non-polar) compounds, forcing aggregation of the hydrophobic compounds. The phenomenon of hydrophobic interactions may be applied to the separation of proteins by using an aqueous salt solution to force a hydrophobic protein to aggregate with or bind adsorptively to hydrophobic functional groups affixed to a solid support. The adsorbed proteins are released from the adsorbent by elution with decreasing salt concentrations, effectively unwinding the environment that promoted the hydrophobic interactions, and leading to loss of hydrophobic interactions between the proteins and the support. The proteins are released from the support in order of increasing hydrophobicity (i.e., the least hydrophobic proteins are released first). Hydrophobic interaction chromatography (HIC) may be distinguished from reverse phase chromatography in that salts are used during the HIC elution step.

In essence, hydrophobic interaction chromatography is a method for separating biomolecules based on the relative strengths of their hydrophobic interactions with a hydrophobic adsorbent. In general, HIC is a selective technique. HIC is sensitive enough to be influenced by non-polar groups typically buried within the tertiary structure of proteins but exposed if the polypeptide chain is incorrectly folded or damaged (e.g., by a protease). This sensitivity can be useful for separating a correctly folded or undamaged protein from other forms.

Hydrophobic interaction chromatography is also a very mild method of separation and purification. The structural damage to a purified biomolecules is minimal, due in part to the stabilizing influence of salts and also to the rather weak interaction with the matrix. Nevertheless, recoveries of purified material are often high. Thus, HIC combines the non-denaturing characteristics of salt precipitation with the precision of chromatography to yield excellent activity recoveries.

Therefore, HIC is a versatile liquid chromatography technique, and should be viewed as a potential component of any purification strategy, often in combination with ion-exchange chromatography and gel filtration. HIC has also found use as an analytical tool in detecting protein conformational changes. HIC requires a minimum of sample pre-treatment and can thus be used effectively in combination with traditional protein precipitation techniques. Protein binding to HIC adsorbents is promoted by moderately high concentrations of anti-chaotropic salts, which also have a stabilizing influence on protein structure.

Most commercially available HIC matrices are in the form of resins. While the resins show high binding capacities for various biological molecules, HIC processes using resins suffer from fouling and low flux capacities. In contrast, chromatography matrices in the form of membranes exhibit increased flux capacities in comparison to their resin counterparts. Therefore, a need exists for a high-binding capacity membrane-based HIC matrix to realize the separation efficiency of a resin combined with the process benefits of a membrane.

### BRIEF SUMMARY OF THE INVENTION

In certain embodiments, the invention relates to a composite material, comprising:

a support member, comprising a plurality of pores extending through the support member; and

a macroporous cross-linked gel, comprising a plurality of macropores, and a plurality of pendant hydrophobic moieties;

wherein the macroporous cross-linked gel is located in the pores of the support member; and the average pore diameter of the macropores is less than the average pore diameter of the pores.

In certain embodiments, the invention relates to any one of the aforementioned composite materials, wherein the macroporous cross-linked gel comprises a polymer derived from acrylamide, N-acryloxysuccinimide, butyl acrylate or methacrylate, N,N-diethylacrylamide, N,N-dimethylacrylamide, 2-(N,N-dimethylamino)ethyl acrylate or methacrylate, 2-(N,N-diethylamino)ethyl acrylate or methacrylate N-[3-(N,N-dimethylamino)propyl]methacrylamide, N,N-dimethylacrylamide, n-dodecyl acrylate, n-dodecyl methacrylate, phenyl acrylate or methacrylate, dodecyl methacrylamide, ethyl acrylate or methacrylate, 2-ethylhexyl acrylate or methacrylate, hydroxypropyl acrylate or methacrylate, glycidyl acrylate or methacrylate, ethylene glycol phenyl ether acrylate or methacrylate, n-heptyl acrylate or methacrylate, 1-hexadecyl acrylate or methacrylate, methacrylamide, methacrylic anhydride, octadecyl acrylamide, octylacrylamide, octyl acrylate or methacrylate, propyl acrylate or methacrylate, N-iso-propylacrylamide, stearyl acrylate or methacrylate, styrene, alkylated styrene derivatives, 4-vinylpyridine, vinylsulfonic acid, N-vinyl-2-pyrrolidinone (VP), acrylamido-2-methyl-1-propanesulfonic acid, styrenesulfonic acid, alginic acid, (3-acrylamidopropyl)trimethylammonium halide, diallyldimethylammonium halide, 4-vinyl-N-methylpyridinium halide, vinylbenzyl-N-trimethylammonium halide, methacryloxyethyltrimethylammonium halide, or 2-(2-methoxy)ethyl acrylate or methacrylate.

In certain embodiments, the invention relates to any one of the aforementioned composite materials, wherein the pendant hydrophobic moieties are ethyl, butyl, hexyl, 2-ethylhexyl, dodecyl, stearyl, hydroxypropyl, phenyl, ether, or poly(propylene glycol) groups.

In certain embodiments, the invention relates to any one of the aforementioned composite materials, wherein the composite material is a membrane.

In certain embodiments, the invention relates to a method, comprising the step of:

contacting a first fluid comprising a substance with any one of the aforementioned composite materials, thereby adsorbing or absorbing a portion of the substance onto the composite material.

In certain embodiments, the invention relates to any one of the aforementioned methods, further comprising the step of:

contacting a second fluid with the substance adsorbed or absorbed onto the composite material, thereby releasing a portion of the substance from the composite material.

In certain embodiments, the invention relates to any one of the aforementioned methods, wherein the macroporous gel displays a specific interaction for the substance.

In certain embodiments, the invention relates to any one of the aforementioned methods, wherein the specific interaction is a hydrophobic interaction.

In certain embodiments, the invention relates to any one of the aforementioned methods, wherein the substance is a biological molecule or biological ion.

In certain embodiments, the invention relates to any one of the aforementioned methods, wherein the biological molecule or biological ion is selected from the group consisting of albumins, lysozyme, viruses, cells, γ-globulins of human and animal origins, immunoglobulins of human and animal origins, proteins of recombinant and natural origins, polypeptides of synthetic and natural origins, interleukin-2 and its receptor, enzymes, monoclonal antibodies, trypsin and its inhibitor, cytochrome C, myoglobin, myoglobulin, α-chymotrypsinogen, recombinant human interleukin, recombinant fusion protein, nucleic acid derived products, DNA of synthetic and natural origins, and RNA of synthetic and natural origins.

In certain embodiments, the invention relates to any one of the aforementioned methods, wherein the first fluid is a buffer.

In certain embodiments, the invention relates to any one of the aforementioned methods, wherein the second fluid is a salt solution.

### BRIEF DESCRIPTION OF THE FIGURES

FIG. 1 depicts graphically the relationship between (i) the ratio (C/C₀) of the concentration of lysozyme in permeate (C) to the concentration of lysozyme in initial sample (C₀) and (ii) lysozyme binding capacity (mg/mL_{membrane}) for three HIC membranes with differing pendant hydrophobic moieties.

FIG. 2 depicts graphically the relationship between (i) the ratio (C/C₀) of the concentration of lysozyme in permeate (C) to the concentration of lysozyme in initial sample (C₀) and (ii) lysozyme binding capacity (mg/mL_{membrane}) for three HIC membranes with pendant butyl moieties and variable levels of gel-matrix hydrophobicity.

FIG. 3 tabulates the binding capacities (mg/mL_{gel/membrane}) (10% breakthrough) for lysozyme and a mAb of: two commercial HIC resins (TOSOH Bioscience); two commercial HIC membranes (Butyl Sepharose 4 Fast Flow, and Sartobind Phenyl); and two HIC membranes (Butyl and Phenyl) of the present invention.

FIG. 4 depicts the separation of myoglobin (1), lysozyme (2) and α-chymotrypsinogen A (3) by hydrophobic interaction chromatography. The proteins were eluted using a gradient buffer change from Buffer A to Buffer B as indicated (gray line, right y-axis) on poly(AAm-co-VP-co-BuMe) membrane prepared as described in Example 5. Peaks are assigned based on individual capture/elution data from Table 2.

FIG. 5 tabulates a summary of various composite materials of the invention and various performance characteristics. In this table, EGDMA is ethylene glycol dimethacrylate, and EGPhA is ethylene glycol phenyl ether acrylate.

### DETAILED DESCRIPTION OF THE INVENTION

### Overview

In certain embodiments, the invention relates to a composite material comprising a macroporous gel within a porous support member. The composite materials are suited for the removal or purification and recovery of hydrophobic solutes, such as proteins and other biomolecules, via adsorption/desorption processes. In certain embodiments, the invention relates to a composite material that is simple and inexpensive to produce.

In certain embodiments, the invention relates to the purification or separation of biomolecules based on differences in surface hydrophobicity. In certain embodiments, biomolecules may be selectively purified in a single step. In certain embodiments, the composite materials demonstrate exceptional performance in comparison to commercially available HIC resins or membranes. In certain embodiments, the composite materials demonstrate comparable performance at higher flow rates than can be achieved with commercially available HIC resins.

### Various Characteristics of Exemplary Composite Materials

Composition of the Macroporous Gels

In certain embodiments, the macroporous gels may be formed through the in situ reaction of one or more polymerizable monomers with one or more cross-linkers. In certain embodiments, the macroporous gels may be formed through the reaction of one or more cross-linkable polymers with one or more cross-linkers. In certain embodiments, a cross-linked gel having macropores of a suitable size may be formed.

The macroporous gel can be selected to comprise hydrophobic monomers. Copolymers of these monomers can be used. A macroporous gel comprising hydrophobic monomers can be used to capture molecules from fluids passing through the pores by hydrophobic interactions. In certain embodiments, the macroporous cross-linked gel comprises a plurality of pendant hydrophobic moieties selected from the group consisting of alkyl, alkenyl, aryl, aralkyl, alkaryl, and aralkoxy groups. In certain embodiments, the alkyl portion may have 1-20 carbon atoms, to which groups of the type hydroxy or halogen may be bound. In certain embodiments, alkyl groups may be branched. In certain embodiments, the branched alkyl functional group may have from 3 to 8 carbon atoms. In certain embodiments, the branched alkyl functional group may contain a sec-carbon, a tert-carbon, or a neo-carbon atom. In certain embodiments, the branched alkyl functional group may be selected from the group consisting of sec-butyl, tert-butyl, tert-pentyl, tert-hexyl, and neopentyl. In certain embodiments, the alkyl group may have from 3 to 8 carbon atoms. In certain embodiments, the alkyl group may have greater than 8 carbon atoms. In certain embodiments, the alkyl group may have greater than 8 carbon atoms and the composite material may still be an effective hydrophobic interaction chromatography medium. This result is contrary to at least one reference that implies that materials having pendant groups longer than C₈ will be ineffective as HIC media, and will function only as reverse phase chromatography media. Hydrophobic Interaction Chromatography: Principles and Methods; Amersham Pharmacia Biotech AB: Uppsala, Sweden, 2000. In certain embodiments, the aryl groups may be phenyl or naphthyl, optionally substituted with one or more nitro groups, halogen atoms, or alkyl groups. In certain embodiments, the pendant hydrophobic moiety may be an acyl group, such as alkanoyl or aroyl, which may contain 2-20 carbon atoms and may be substituted with one or more halogen atoms, nitro, or hydroxy groups. In certain embodiments, the pendant hydrophobic moiety may be an aroyl group, such as benzoyl, chlorbenzoyl, naphthoyl, or nitro benzoyl.

In certain embodiments, suitable polymerizable monomers include monomers containing vinyl or acryl groups. In certain embodiments, polymerizable monomers may be selected from the group consisting of acrylamide, N-acryloxysuccinimide, butyl acrylate and methacrylate, N,N-diethylacrylamide, N,N-dimethylacrylamide, 2-(N,N-dimethylamino)ethyl acrylate and methacrylate, N-[3-(N,N-dimethylamino)propyl]methacrylamide, N,N-dimethylacrylamide, n-dodecyl acrylate, n-dodecyl methacrylate, phenyl acrylate and methacrylate, dodecyl methacrylamide, ethyl acrylate and methacrylate, 2-ethylhexyl methacrylate, hydroxypropyl methacrylate, glycidyl acrylate and methacrylate, ethylene glycol phenyl ether acrylate or methacrylate, n-heptyl acrylate and methacrylate, 1-hexadecyl acrylate and methacrylate, methacrylamide, methacrylic anhydride, octadecyl acrylamide, octylacrylamide, octyl methacrylate, propyl acrylate and methacrylate, N-iso-propylacrylamide, stearyl acrylate and methacrylate, styrene, alkylated styrene derivatives, 4-vinylpyridine, vinylsulfonic acid, and N-vinyl-2-pyrrolidinone (VP). In certain embodiments, the polymerizable monomers may comprise butyl, hexyl, phenyl, ether, or poly(propylene glycol) side chains. In certain embodiments, various other vinyl or acryl monomers comprising a reactive functional group may be used; these reactive monomers may be subsequently functionalized with a hydrophobic moiety.

In certain embodiments, suitable monomers may be selected based on their partition coefficients. A partition coefficient (log P value) is the ratio of the equilibrium concentrations of an un-ionized compound between two immiscible solvents. In other words, the coefficients are an estimation of differential solubility of the compound between the two solvents. In certain embodiments, a portion of the monomers used in the preparation of the inventive composite materials are hydrophobic. For example, ethyl acrylate has an estimated log P (octanol-water) of about 1.2, phenyl acrylate has an estimated log P (octanol-water) of about 1.9, and lauryl methacrylate has an estimated log P (octanol-water) of about 6.7. In certain embodiments, the composite materials of the present invention may comprise a polymer derived from a monomer with a log P value (octanol-water) from about 1 to about 7. In certain embodiments, a first monomer may be copolymerized with a second monomer, wherein the first monomer has a log P value (octanol-water) from about 1 to about 7; and the second monomer has a log P value (octanol-water) from about −1 to about 1. In certain embodiments, the molar ratio of first monomer to second monomer may be from about 0.01:1 to about 1:1. In certain embodiments, the molar ratio of first monomer to second monomer may be from about 0.05:1 to about 0.5:1. In certain embodiments, the molar ratio of first monomer to second monomer may be about 0.1:1, about 0.15:1, or about 0.20:1. Exemplary monomers and their estimated log P values (octanol-water) are provided in Table 1.

TABLE 1
Estimated log P values (octanol-water) of various monomers

| Monomer | log P_{octanol/water} |
|---|---|
| acrylamide | −0.8 |
| N-vinyl-2-pyrrolidinone | 0.2 |
| 2-hydroxyethyl methacrylate | 0.3 |
| methyl acrylate | 0.7 |
| glycidyl methacrylate | 0.8 |
| ethyl acrylate | 1.2 |
| methyl methacrylate | 1.3 |
| phenyl acrylate | 1.9 |
| n-butyl acrylate | 2.2 |
| n-butyl methacrylate | 2.8 |
| n-hexyl acrylate | 3.2 |
| 2-ethylhexyl acrylate | 4.1 |
| n-octyl acrylate | 4.2 |
| n-decyl acrylate | 5.2 |
| lauryl acrylate | 6.1 |
| lauryl methacrylate | 6.7 |

In certain embodiments, the monomer may comprise a reactive functional group. In certain embodiments, the reactive functional group of the monomer may be reacted with any of a variety of specific ligands. In certain embodiments, the reactive functional group of the monomer may be reacted with a hydrophobic moiety. In certain embodiments, this technique allows for partial or complete control of ligand density or pore size. In certain embodiments, the functionalization of the monomer with a hydrophobic moiety imparts further hydrophobic character to the resulting gel. In certain embodiments, the reactive functional group of the monomer may be functionalized prior to the gel-forming reaction. In certain embodiments, the reactive functional group of the monomer may be functionalized subsequent to the gel-forming reaction. For example, if the monomer is glycidyl methacrylate, the epoxide functionality of the monomer may be reacted with butyl amine to introduce butyl functionality into the resultant polymer. In certain embodiments, monomers, such as glycidyl methacrylate, acrylamidoxime, acrylic anhydride, azelaic anhydride, maleic anhydride, hydrazide, acryloyl chloride, 2-bromoethyl methacrylate, or vinyl methyl ketone, may be further functionalized. In certain embodiments, if this technique is used, suitable monomers are not identified by their log P values, but by the overall hydrophobicity of the resultant polymer after functionalization.

In certain embodiments, the cross-linking agent may be a compound containing at least two vinyl or acryl groups. In certain embodiments, the cross-linking agent may be selected from the group consisting of bisacrylamidoacetic acid, 2,2-bis[4-(2-acryloxyethoxy)phenyl]propane, 2,2-bis(4-methacryloxyphenyl)propane, butanediol diacrylate and dimethacrylate, 1,4-butanediol divinyl ether, 1,4-cyclohexanediol diacrylate and dimethacrylate, 1,10-dodecanediol diacrylate and dimethacrylate, 1,4-diacryloylpiperazine, diallylphthalate, 2,2-dimethylpropanediol diacrylate and dimethacrylate, dipentaerythritol pentaacrylate, dipropylene glycol diacrylate and dimethacrylate, N,N-dodecamethylenebisacrylamide, divinylbenzene, glycerol trimethacrylate, glycerol tris(acryloxypropyl)ether, N,N′-hexamethylenebisacrylamide, N,N′-octamethylenebisacrylamide, 1,5-pentanediol diacrylate and dimethacrylate, 1,3-phenylenediacrylate, poly(ethylene glycol) diacrylate and dimethacrylate, poly(propylene) diacrylate and dimethacrylate, triethylene glycol diacrylate and dimethacrylate, triethylene glycol divinyl ether, tripropylene glycol diacrylate or dimethacrylate, diallyl diglycol carbonate, poly(ethylene glycol) divinyl ether, N,N′-dimethacryloylpiperazine, divinyl glycol, ethylene glycol diacrylate, ethylene glycol dimethacrylate, N,N′-methylenebisacrylamide, 1,1,1-trimethylolethane trimethacrylate, 1,1,1-trimethylolpropane triacrylate, 1,1,1-trimethylolpropane trimethacrylate (TRIM-M), vinyl acrylate, 1,6-hexanediol diacrylate and dimethacrylate, 1,3-butylene glycol diacrylate and dimethacrylate, alkoxylated cyclohexane dimethanol diacrylate, alkoxylated hexanediol diacrylate, alkoxylated neopentyl glycol diacrylate, aromatic dimethacrylate, caprolactone modified neopentylglycol hydroxypivalate diacrylate, cyclohexane dimethanol diacrylate and dimethacrylate, ethoxylated bisphenol diacrylate and dimethacrylate, neopentyl glycol diacrylate and dimethacrylate, ethoxylated trimethylolpropane triarylate, propoxylated trimethylolpropane triacrylate, propoxylated glyceryl triacrylate, pentaerythritol triacrylate, tris(2-hydroxyethyl)isocyanurate triacrylate, di-trimethylolpropane tetraacrylate, dipentaerythritol pentaacrylate, ethoxylated pentaerythritol tetraacrylate, pentaacrylate ester, pentaerythritol tetraacrylate, caprolactone modified dipentaerythritol hexaacrylate, N,N′,-methylenebisacrylamide, diethylene glycol diacrylate and dimethacrylate, trimethylolpropane triacrylate, ethylene glycol diacrylate and dimethacrylate, tetra(ethylene glycol) diacrylate, 1,6-hexanediol diacrylate, divinylbenzene, and poly(ethylene glycol) diacrylate.

In certain embodiments, the size of the macropores in the resulting gel increases as the concentration of cross-linking agent is increased. For example, the molar ratio of cross-linking agent to monomer(s) may be in the range from about 5:95 to about 70:30, in the range from about 10:90 to about 50:50, or in the range from about 15:85 to about 45:55. In certain embodiments, the molar ratio of cross-linking agent to monomer(s) may be about 10%, about 11%, about 12%, about 13%, about 14%, about 15%, about 16%, about 17%, about 18%, about 19%, about 20%, about 21%, about 22%, about 23%, about 24%, or about 25%.

In certain embodiments, the properties of the composite materials may be tuned by adjusting the average pore diameter of the macroporous gel. The size of the macropores is generally dependent on the nature and concentration of the cross-linking agent, the nature of the solvent or solvents in which the gel is formed, the amount of any polymerization initiator or catalyst and, if present, the nature and concentration of porogen. In certain embodiments, the composite material may have a narrow pore-size distribution.

Porous Support Member

In some embodiments, the porous support member is made of polymeric material and contains pores of average size between about 0.1 and about 25 μm, and a volume porosity between about 40% and about 90%. Many porous substrates or membranes can be used as the support member but the support may be a polymeric material. In certain embodiments, the support may be a polyolefin, which is available at low cost. In certain embodiments, the polyolefin may be poly(ethylene), poly(propylene), or poly(vinylidene difluoride). Extended polyolefin membranes made by thermally induced phase separation (TIPS), or non-solvent induced phase separation are mentioned. In certain embodiments, the support member may be made from natural polymers, such as cellulose or its derivatives. In certain embodiments, suitable supports include polyethersulfone membranes, poly(tetrafluoroethylene) membranes, nylon membranes, cellulose ester membranes, or filter papers.

In certain embodiments, the porous support is composed of woven or non-woven fibrous material, for example, a polyolefin such as polypropylene. Such fibrous woven or non-woven support members can have pore sizes larger than the TIPS support members, in some instances up to about 75 μm. The larger pores in the support member permit formation of composite materials having larger macropores in the macroporous gel. Non-polymeric support members can also be used, such as ceramic-based supports. The porous support member can take various shapes and sizes.

In some embodiments, the support member is in the form of a membrane that has a thickness from about 10 to about 2000 μm, from about 10 to about 1000 μm, or from about 10 to about 500 μm. In other embodiments, multiple porous support units can be combined, for example, by stacking. In one embodiment, a stack of porous support membranes, for example, from 2 to 10 membranes, can be assembled before the macroporous gel is formed within the void of the porous support. In another embodiment, single support member units are used to form composite material membranes, which are then stacked before use.

Relationship Between Macroporous Gel and Support Member

The macroporous gel may be anchored within the support member. The term “anchored” is intended to mean that the gel is held within the pores of the support member, but the term is not necessarily restricted to mean that the gel is chemically bound to the pores of the support member. The gel can be held by the physical constraint imposed upon it by enmeshing and intertwining with structural elements of the support member, without actually being chemically grafted to the support member, although in some embodiments, the macroporous gel may be grafted to the surface of the pores of the support member.

Because the macropores are present in the gel that occupies the pores of the support member, the macropores of the gel must be smaller than the pores of the support member. Consequently, the flow characteristics and separation characteristics of the composite material are dependent on the characteristics of the macroporous gel, but are largely independent of the characteristics of the porous support member, with the proviso that the size of the pores present in the support member is greater than the size of the macropores of the gel. The porosity of the composite material can be tailored by filling the support member with a gel whose porosity is partially or completely dictated by the nature and amounts of monomer or polymer, cross-linking agent, reaction solvent, and porogen, if used. As pores of the support member are filled with the same macroporous gel material, a high degree of consistency is achieved in properties of the composite material, and for a particular support member these properties are determined partially, if not entirely, by the properties of the macroporous gel. The net result is that the invention provides control over macropore-size, permeability and surface area of the composite materials.

The number of macropores in the composite material is not dictated by the number of pores in the support material. The number of macropores in the composite material can be much greater than the number of pores in the support member because the macropores are smaller than the pores in the support member. As mentioned above, the effect of the pore-size of the support material on the pore-size of the macroporous gel is generally negligible. An exception is found in those cases where the support member has a large difference in pore-size and pore-size distribution, and where a macroporous gel having very small pore-sizes and a narrow range in pore-size distribution is sought. In these cases, large variations in the pore-size distribution of the support member are weakly reflected in the pore-size distribution of the macroporous gel. In certain embodiments, a support member with a somewhat narrow pore-size range may be used in these situations.

### Preparation of Composite Materials

In certain embodiments, the composite materials of the invention may be prepared by single-step methods. In certain embodiments, these methods may use water or other environmentally benign solvents as the reaction solvent. In certain embodiments, the methods may be rapid and, therefore, may lead to easier manufacturing processes.

In certain embodiments, the composite materials of the invention may be prepared by mixing one or more monomers, one or more cross-linking agents, one or more initiators, and optionally one or more porogens, in one or more suitable solvents. In certain embodiments, the resulting mixture may be homogeneous. In certain embodiments, the mixture may be heterogeneous. In certain embodiments, the mixture may then be introduced into a suitable porous support, where a gel forming reaction may take place.

In certain embodiments, suitable solvents for the gel-forming reaction include 1,3-butanediol, di(propylene glycol) propyl ether, N,N-dimethylacetamide, di(propylene glycol) methyl ether acetate (DPMA), water, dioxane, dimethylsulfoxide (DMSO), dimethylformamide (DMF), acetone, ethanol, N-methylpyrrolidone (NMP), tetrahydrofuran (THF), ethyl acetate, acetonitrile, toluene, xylenes, hexane, N-methylacetamide, propanol, methanol, or mixtures thereof. In certain embodiments, solvents that have a higher boiling point may be used, as these solvents reduce flammability and facilitate manufacture. In certain embodiments, solvents that have a low toxicity may be used, so they may be readily disposed of after use. An example of such a solvent is dipropyleneglycol monomethyl ether (DPM).

In certain embodiments, a porogen may be added to the reactant mixture, wherein porogens may be broadly described as pore-generating additives. In certain embodiments, the porogen may be selected from the group consisting of thermodynamically poor solvents and extractable polymers, for example, poly(ethyleneglycol), surfactants, and salts.

In some embodiments, components of the gel forming reaction react spontaneously at room temperature to form the macroporous gel. In other embodiments, the gel forming reaction must be initiated. In certain embodiments, the gel forming reaction may be initiated by any known method, for example, through thermal activation or UV radiation. In certain embodiments, the reaction may be initiated by UV radiation in the presence of a photoinitiator. In certain embodiments, the photoinitiator may be selected from the group consisting of 2-hydroxy-1-[4-2(hydroxyethoxy)phenyl]-2-methyl-1-propanone (Irgacure® 2959), 2,2-dimethoxy-2-phenylacetophenone (DMPA), benzophenone, benzoin and benzoin ethers, such as benzoin ethyl ether and benzoin methyl ether, dialkoxyacetophenones, hydroxyalkylphenones, and α-hydroxymethyl benzoin sulfonic esters. Thermal activation may require the addition of a thermal initiator. In certain embodiments, the thermal initiator may be selected from the group consisting of 1,1′-azobis(cyclohexanecarbonitrile) (VAZO® catalyst 88), azobis(isobutyronitrile) (AIBN), potassium persulfate, ammonium persulfate, and benzoyl peroxide.

In certain embodiments, the gel-forming reaction may be initiated by UV radiation. In certain embodiments, a photoinitiator may be added to the reactants of the gel forming reaction, and the support member containing the mixture of monomer, cross-linking agent, and photoinitiator may be exposed to UV radiation at wavelengths from about 250 nm to about 400 nm for a period of a few seconds to a few hours. In certain embodiments, the support member containing the mixture of monomer, cross-linking agent, and photoinitiator may be exposed to UV radiation at about 350 nm for a period of a few seconds to a few hours. In certain embodiments, the support member containing the mixture of monomer, cross-linking agent, and photoinitiator may be exposed to UV radiation at about 350 nm for about 10 minutes. In certain embodiments, visible wavelength light may be used to initiate the polymerization. In certain embodiments, the support member must have a low absorbance at the wavelength used so that the energy may be transmitted through the support member.

In certain embodiments, the rate at which polymerization is carried out may have an effect on the size of the macropores obtained in the macroporous gel. In certain embodiments, when the concentration of cross-linker in a gel is increased to sufficient concentration, the constituents of the gel begin to aggregate to produce regions of high polymer density and regions with little or no polymer, which latter regions are referred to as “macropores” in the present specification. This mechanism is affected by the rate of polymerization. In certain embodiments, the polymerization may be carried out slowly, such as when a low light intensity in the photopolymerization is used. In this instance, the aggregation of the gel constituents has more time to take place, which leads to larger pores in the gel. In certain embodiments, the polymerization may be carried out at a high rate, such as when a high intensity light source is used. In this instance, there may be less time available for aggregation and smaller pores are produced.

In certain embodiments, once the composite materials are prepared, they may be washed with various solvents to remove any unreacted components and any polymer or oligomers that are not anchored within the support. In certain embodiments, solvents suitable for the washing of the composite material include water, acetone, methanol, ethanol, and DMF.

### Exemplary Uses of the Composite Materials

In certain embodiments, the invention relates to a method, wherein a fluid is passed through the macropores of the macroporous cross-linked gel of any one of the aforementioned composite materials. By tailoring the conditions for fractionation, good selectivity, even for substances of the same size, can be obtained.

In certain embodiments, the invention relates to a method of separating biomolecules, such as proteins or immunoglobulins, from solution based on specific interactions the biomolecules have with the composite materials. In certain embodiments, the invention relates to a method of purifying biomolecules such as proteins or immunoglobulins. In certain embodiments, the invention relates to a method of purifying proteins or monoclonal antibodies with high selectivity. In certain embodiments, the invention relates to a method, wherein the biological molecule or biological ion retains its tertiary or quaternary structure, which may be important in retaining biological activity. In certain embodiments, biological molecules or biological ions that may be separated or purified include proteins such as albumins, e.g., bovine serum albumin, and lysozyme. In certain embodiments, biological molecules or biological ions that may be separated include γ-globulins of human and animal origins, immunoglobulins such as IgG, IgM, or IgE of human and animal origins, proteins of recombinant and natural origin including protein A, phytochrome, halophilic protease, poly(3-hydroxybutyrate)depolymerase, aculaecin-A acylase, polypeptides of synthetic and natural origin, interleukin-2 and its receptor, enzymes such as phosphatase, dehydrogenase, ribonuclease A, etc., monoclonal antibodies, fragments of antibodies, trypsin and its inhibitor, albumins of varying origins, e.g., α-lactalbumin, human serum albumin, chicken egg albumin, ovalbumin etc., cytochrome C, immunoglobulins, myoglobulin, recombinant human interleukin, recombinant fusion protein, nucleic acid derived products, DNA and RNA of synthetic and natural origin, DNA plasmids, lectin, α-chymotrypsinogen, and natural products including small molecules. In certain embodiments, the invention relates to a method of recovering an antibody fragment from variants, impurities, or contaminants associated therewith. In certain embodiments, biomolecule separation or purification may occur substantially in the macropores of the macroporous cross-linked gel.

In certain embodiments, the invention relates to a method of reversible adsorption of a substance. In certain embodiments, an adsorbed substance may be released by changing the liquid that flows through the macroporous gel. In certain embodiments, the uptake and release of substances may be controlled by variations in the composition of the macroporous cross-linked gel.

In certain embodiments, the invention relates to a method, wherein the substance may be applied to the composite material from a buffered solution. In certain embodiments, the buffer is sodium phosphate. In certain embodiments, the concentration of the buffer may be about 25 mM, about 50 mM, about 0.1 M, or about 0.2 M. In certain embodiments, the pH of the buffered solution is about 4, about 5, about 6, about 7, about 8, or about 9.

In certain embodiments, the invention relates to a method, wherein the substance may be eluted using varying concentrations of aqueous salt solutions. In certain embodiments, the salt is selected from the group consisting of (NH₄)₂SO₄, K₂SO₄, glycine-HCl, phosphate, citric acid, sodium citrate, NaCl, Na₂SO₄, NaPO₄, sodium acetate, and NH₄Cl. In certain embodiments, the salt concentration may range from about 3.0 M to about 0.2 M. In certain embodiments, the salt concentration may be about 3.0 M, about 2.8 M, about 2.6 M, about 2.4 M, about 2.2 M, about 2.0 M, about 1.8 M, about 1.6 M, about 1.4 M, about 1.2 M, about 1.0 M, about 0.8 M, about 0.6 M, about 0.4 M, or about 0.2 M.

In certain embodiments, the invention relates to a method that exhibits high binding capacities. In certain embodiments, the invention relates to a method that exhibits binding capacities of about 10 mg/mL_{membrane}, about 15 mg/mL_{membrane}, about 20 mg/mL_{membrane}, about 25 mg/mL_{membrane}, about 30 mg/mL_{membrane}, about 35 mg/mL_{membrane}, about 40 mg/mL_{membrane}, about 45, mg/mL_{membrane}, or about 50 mg/mL_{membrane} at 10% breakthrough.

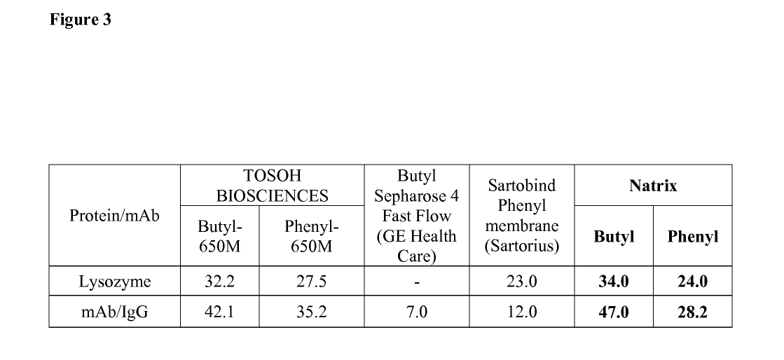

In certain embodiments, methods of the invention result in binding capacities comparable to or higher than those reported with the use of conventional HIC resins. However, the inventive methods may be run at a significantly higher flow rates, due to convective flow, than the flow rates achieved in methods using HIC resins. In certain embodiments, the methods of the present invention do not suffer from the problematic pressure drops associated with methods using HIC resins. In certain embodiments, the methods of the present invention also allow for higher binding capacities than those reported for commercially-available HIC membranes (see, e.g., FIG. 3).

In certain embodiments, the flow rate during binding (the first flow rate) may be from about 0.1 to about 10 mL/min. In certain embodiments, the flow rate during elution (the second flow rate) may be from about 0.1 to about 10 mL/min. In certain embodiments, the first flow rate or the second flow rate may be about 0.1 mL/min, about 0.5 mL/min, about 1.0 mL/min, about 1.5 mL/min, about 2.0 mL/min, about 2.5 mL/min, about 3.0 mL/min, about 4.0 mL/min, about 4.5 mL/min, about 5.0 mL/min, about 5.5 mL/min, about 6.0 mL/min, about 6.5 mL/min, about 7.0 mL/min, about 7.5 mL/min, about 8.0 mL/min, about 8.5 mL/min, about 9.0 mL/min, about 9.5 mL/min, or about 10.0 mL/min. In certain embodiments, the first flow rate or the second flow rate may be from about 0.5 mL/min to about 5.0 mL/min.

The water flux, Q_{H2O} (kg/m²h), was calculated using the following equation:

$${Q}_{{H}_{2} O} = \frac{( {m}_{1} - {m}_{2} )}{A \cdot t}$$

where m₁ is the mass of container with the water sample, m₂ is the mass of container, A is the active membrane surface area (38.5 cm²) and t is the time.

The hydrodynamic Darcy permeability, k (m²) of the membrane can be calculated from the following equation:

$$k = \frac{{Q}_{{H}_{2} O} ηδ}{3600 {d}_{{H}_{2} O} Δ P}$$

where η is the water viscosity (Pa·s), δ is the membrane thickness (m), d_{H2O} is the water density (kg/m³), and ΔP (Pa) is the pressure difference at which the flux, Q_{H2O}, was measured.

The hydrodynamic Darcy permeability of the membrane may be used to estimate an average hydrodynamic radius of the pores in the porous gel. The hydrodynamic radius, rₕ, is defined as the ratio of the pore volume to the pore wetted surface area and can be obtained from the Carman-Kozeny equation given in the book by J. Happel and H. Brenner, Low Reynolds Number Hydrodynamics, Noordhof Int. Publ., Leyden, 1973, p. 393:

$$k = \frac{ɛ {r}_{h}^{2}}{K}$$

where K is the Kozeny constant and ε is the membrane porosity. The Kozeny constant K≠5 for porosity 0.5<ε<0.7. The porosity of the membrane can be estimated from porosity of the support by subtracting the volume of the gel polymer.

In certain embodiments, an additive may be added to the eluting salt solution (the second fluid, or the third or later fluid). In certain embodiments, the additive is added in a low concentration (e.g., less than about 1 M, about 0.5 M, or about 0.2 M). In certain embodiments, the additive is a water-miscible alcohol, a detergent, dimethyl sulfoxide, dimethyl formamide, or an aqueous solution of a chaotropic salt. In certain embodiments, not wishing to be bound by any particular theory, the additive may decrease the surface tension of water, thus weakening the hydrophobic interactions to give a subsequent dissociation of the ligand-solute complex.

In certain embodiments, the methods of the invention may be a subsequent step in the purification of materials that have been precipitated with ammonium sulfate or eluted in high salt concentrations during ion-exchange chromatography. In certain embodiments, the methods of the invention may be used subsequent to an affinity chromatography step. In certain embodiments, the methods of the invention may be an intermediate purification step that separates the correctly folded form of a biomolecule, such as a growth factor (e.g., IGF-1), from a misfolded, yet stable form, which might be generated in a refolding process.

In certain embodiments, the invention relates to a one-step method of biomolecule purification. In certain embodiments, the invention relates to a method of biomolecule separation that is easier to scale-up, is less labor intensive, is faster, and has lower capital costs than the commonly used conventional packed-column chromatography techniques.

### Exemplary Composite Materials

In certain embodiments, the invention relates to a composite material, comprising:

a support member, comprising a plurality of pores extending through the support member; and

a macroporous cross-linked gel, comprising a plurality of macropores, and a plurality of pendant hydrophobic moieties;

wherein the macroporous cross-linked gel is located in the pores of the support member; and the average pore diameter of the macropores is less than the average pore diameter of the pores.

In certain embodiments, the invention relates to any one of the aforementioned composite materials, wherein the macroporous cross-linked gel comprises a polymer derived from acrylamide, N-acryloxysuccinimide, butyl acrylate or methacrylate, N,N-diethylacrylamide, N,N-dimethylacrylamide, 2-(N,N-dimethylamino)ethyl acrylate or methacrylate, 2-(N,N-diethylamino)ethyl acrylate or methacrylate N-[3-(N,N-dimethylamino)propyl]methacrylamide, N,N-dimethylacrylamide, n-dodecyl acrylate, n-dodecyl methacrylate, phenyl acrylate or methacrylate, dodecyl methacrylamide, ethyl acrylate or methacrylate, 2-ethylhexyl acrylate or methacrylate, hydroxypropyl acrylate or methacrylate, glycidyl acrylate or methacrylate, ethylene glycol phenyl ether acrylate or methacrylate, n-heptyl acrylate or methacrylate, 1-hexadecyl acrylate or methacrylate, methacrylamide, methacrylic anhydride, octadecyl acrylamide, octylacrylamide, octyl acrylate or methacrylate, propyl acrylate or methacrylate, N-iso-propylacrylamide, stearyl acrylate or methacrylate, styrene, alkylated styrene derivatives, 4-vinylpyridine, vinylsulfonic acid, N-vinyl-2-pyrrolidinone (VP), acrylamido-2-methyl-1-propanesulfonic acid, styrene sulfonic acid, alginic acid, (3-acrylamidopropyl)trimethylammonium halide, diallyldimethylammonium halide, 4-vinyl-N-methylpyridinium halide, vinylbenzyl-N-trimethylammonium halide, methacryloxyethyltrimethylammonium halide, or 2-(2-methoxy)ethyl acrylate or methacrylate. In certain embodiments, the halide is chloride, bromide, or iodide.

In certain embodiments, the invention relates to any one of the aforementioned composite materials, wherein the macroporous cross-linked gel comprises a polymer derived from acrylamide, butyl acrylate or methacrylate, n-dodecyl acrylate, n-dodecyl methacrylate, phenyl acrylate or methacrylate, ethyl acrylate or methacrylate, 2-ethylhexyl methacrylate, hydroxypropyl methacrylate, glycidyl acrylate or methacrylate, ethylene glycol phenyl ether acrylate or methacrylate, n-heptyl acrylate or methacrylate, 1-hexadecyl acrylate or methacrylate, octyl acrylate or methacrylate, propyl acrylate or methacrylate, stearyl acrylate or methacrylate, or N-vinyl-2-pyrrolidinone (VP).

In certain embodiments, the invention relates to any one of the aforementioned composite materials, wherein the pendant hydrophobic moieties are ethyl, butyl, hexyl, 2-ethylhexyl, dodecyl, stearyl, hydroxypropyl, phenyl, ether, or poly(propylene glycol) groups.

In certain embodiments, the invention relates to any one of the aforementioned composite materials, wherein the macroporous cross-linked gel comprises a polymer derived from a monomer with a log P value (octanol-water) from about 1 to about 7. In certain embodiments, the invention relates to any one of the aforementioned composite materials, wherein the macroporous cross-linked gel comprises a polymer derived from a monomer with a log P value (octanol-water) of about 1, about 1.5, about 2, about 2.5, about 3, about 3.5, about 4, about 4.5, about 5, about 5.5, about 6, about 6.5, or about 7.

In certain embodiments, the invention relates to any one of the aforementioned composite materials, wherein the macroporous cross-linked gel comprises a polymer derived from a first monomer and a second monomer, the first monomer has a log P value (octanol-water) from about 1 to about 7; and the second monomer has a log P value (octanol-water) from about −1 to about 1.

In certain embodiments, the invention relates to any one of the aforementioned composite materials, wherein the molar ratio of the first monomer to the second monomer is about 0.01:1 to about 1:1.

In certain embodiments, the invention relates to any one of the aforementioned composite materials, wherein the molar ratio of the first monomer to the second monomer is about 0.05:1 to about 0.5:1.

In certain embodiments, the invention relates to any one of the aforementioned composite materials, wherein the molar ratio of the first monomer to the second monomer is about 0.1:1, about 0.15:1, or about 0.20:1.

In certain embodiments, the invention relates to any one of the aforementioned composite materials wherein the macroporous cross-linked gel comprises macropores; the macroporous cross-linked gel has a volume porosity from about 30% to about 80%; and the macropores have an average pore diameter from about 10 nm to about 3000 nm.

In certain embodiments, the invention relates to any one of the aforementioned composite materials wherein the macroporous cross-linked gel comprises macropores; the macroporous cross-linked gel has a volume porosity from about 40% to about 70%. In certain embodiments, the invention relates to any one of the aforementioned composite materials wherein the macroporous cross-linked gel comprises macropores; the macroporous cross-linked gel has a volume porosity of about 40%, about 45%, about 50%, about 55%, about 60%, about 65%, or about 70%.

In certain embodiments, the invention relates to any one of the aforementioned composite materials, wherein the average pore diameter of the macropores is about 25 nm to about 1500 nm.

In certain embodiments, the invention relates to any one of the aforementioned composite materials, wherein the average pore diameter of the macropores is about 50 nm to about 1000 nm. In certain embodiments, the invention relates to any one of the aforementioned composite materials, wherein the average pore diameter of the macropores is about 50 nm, about 100 nm, about 150 nm, about 200 nm, about 250 nm, about 300 nm, about 350 nm, about 400 nm, about 450 nm, about 500 nm, about 550 nm, about 600 nm, about 650 nm, or about 700 nm.

In certain embodiments, the invention relates to any one of the aforementioned composite materials, wherein the average pore diameter of the macropores is from about 300 nm to about 400 nm.

In certain embodiments, the invention relates to any one of the aforementioned composite materials, wherein the composite material is a membrane.

In certain embodiments, the invention relates to any one of the aforementioned composite materials, wherein the support member has a void volume; and the void volume of the support member is substantially filled with the macroporous cross-linked gel.

In certain embodiments, the invention relates to any one of the aforementioned composite materials, wherein the support member comprises a polymer; the support member is about 10 μm to about 500 μm thick; the pores of the support member have an average pore diameter from about 0.1 μm to about 25 μm; and the support member has a volume porosity from about 40% to about 90%.

In certain embodiments, the invention relates to any one of the aforementioned composite materials, wherein the support member is about 10 μm to about 500 μm thick. In certain embodiments, the invention relates to any one of the aforementioned composite materials, wherein the support member is about 30 μm to about 300 μm thick. In certain embodiments, the invention relates to any one of the aforementioned composite materials, wherein the support member is about 30 μm, about 50 μm, about 100 μm, about 150 μm, about 200 μm, about 250 μm, or about 300 μm thick.

In certain embodiments, the invention relates to any one of the aforementioned composite materials, wherein the pores of the support member have an average pore diameter from about 0.1 μm to about 25 μm. In certain embodiments, the invention relates to any one of the aforementioned composite materials, wherein the pores of the support member have an average pore diameter from about 0.5 μm to about 15 μm. In certain embodiments, the invention relates to any one of the aforementioned composite materials, wherein the pores of the support member have an average pore diameter of about 0.5 μm, about 1 μm, about 2 μm, about 3 μm, about 4 μm, about 5 μm, about 6 μm, about 7 μm, about 8 μm, about 9 μm, about 10 μm, about 11 μm, about 12 μm, about 13 μm, about 14 μm, or about 15 μm.

In certain embodiments, the invention relates to any one of the aforementioned composite materials, wherein the support member has a volume porosity from about 40% to about 90%. In certain embodiments, the invention relates to any one of the aforementioned composite materials, wherein the support member has a volume porosity from about 50% to about 80%. In certain embodiments, the invention relates to any one of the aforementioned composite materials, wherein the support member has a volume porosity of about 50%, about 60%, about 70%, or about 80%.

In certain embodiments, the invention relates to any one of the aforementioned composite materials, wherein the support member comprises a polyolefin.

In certain embodiments, the invention relates to any one of the aforementioned composite materials, wherein the support member comprises a polymeric material selected from the group consisting of polysulfones, polyethersulfones, polyphenyleneoxides, polycarbonates, polyesters, cellulose and cellulose derivatives.

In certain embodiments, the invention relates to any one of the aforementioned composite materials, wherein the support member comprises a fibrous woven or non-woven fabric comprising a polymer; the support member is from about 10 μm to about 2000 μm thick; the pores of the support member have an average pore diameter of from about 0.1 μm to about 25 μm; and the support member has a volume porosity from about 40% to about 90%.

### Exemplary Methods

In certain embodiments, the invention relates to a method, comprising the step of:

contacting at a first flow rate a first fluid comprising a substance with any one of the aforementioned composite materials, thereby adsorbing or absorbing a portion of the substance onto the composite material.

In certain embodiments, the invention relates to any one of the aforementioned methods, wherein the fluid flow path of the first fluid is substantially perpendicular to the pores of the support member.

In certain embodiments, the invention relates to any one of the aforementioned methods, wherein the fluid flow path of the first fluid is substantially through the macropores of the composite material.

In certain embodiments, the invention relates to any one of the aforementioned methods, further comprising the step of:

contacting at a second flow rate a second fluid with the substance adsorbed or absorbed onto the composite material, thereby releasing a portion of the substance from the composite material.

In certain embodiments, the invention relates to any one of the aforementioned methods, wherein the fluid flow path of the second fluid is substantially perpendicular to the pores of the support member.

In certain embodiments, the invention relates to any one of the aforementioned methods, wherein the fluid flow path of the second fluid is substantially through the macropores of the composite material.

In certain embodiments, the invention relates to any one of the aforementioned methods, wherein the macroporous gel displays a specific interaction for the substance.

In certain embodiments, the invention relates to any one of the aforementioned methods, wherein the specific interaction is a hydrophobic interaction.

In certain embodiments, the invention relates to any one of the aforementioned methods, wherein the substance is a biological molecule or biological ion.

In certain embodiments, the invention relates to any one of the aforementioned methods, wherein the biological molecule or biological ion is selected from the group consisting of albumins, lysozyme, viruses, cells, γ-globulins of human and animal origins, immunoglobulins of human and animal origins, proteins of recombinant and natural origins, polypeptides of synthetic and natural origins, interleukin-2 and its receptor, enzymes, monoclonal antibodies, trypsin and its inhibitor, cytochrome C, myoglobin, myoglobulin, α-chymotrypsinogen, recombinant human interleukin, recombinant fusion protein, nucleic acid derived products, DNA of synthetic and natural origins, and RNA of synthetic and natural origins.

In certain embodiments, the invention relates to any one of the aforementioned methods, wherein the biological molecule or biological ion is lysozyme, hIgG, myoglobin, human serum albumin, soy trypsin inhibitor, transferring, enolase, ovalbumin, ribonuclease, egg trypsin inhibitor, cytochrome c, Annexin V, or α-chymotrypsinogen.

In certain embodiments, the invention relates to any one of the aforementioned methods, wherein the first fluid is a buffer. In certain embodiments, the invention relates to any one of the aforementioned methods, wherein the concentration of the buffer in the first fluid is about 25 mM, about 50 mM, about 0.1 M, or about 0.2 M. In certain embodiments, the invention relates to any one of the aforementioned methods, wherein the pH of the first fluid is about 4, about 5, about 6, about 7, about 8, or about 9.

In certain embodiments, the invention relates to any one of the aforementioned methods, wherein the second fluid is a salt solution. In certain embodiments, the invention relates to any one of the aforementioned methods, wherein the salt is selected from the group consisting of (NH₄)₂SO₄, K₂SO₄, glycine-HCl, phosphate, citric acid, sodium citrate, NaCl, Na₂SO₄, NaPO₄, sodium acetate, and NH₄Cl. In certain embodiments, the invention relates to any one of the aforementioned methods, wherein the salt concentration in the second fluid is from about 3.0 M to about 0.2 M. In certain embodiments, the invention relates to any one of the aforementioned methods, wherein the salt concentration is about 3.0 M, about 2.8 M, about 2.6 M, about 2.4 M, about 2.2 M, about 2.0 M, about 1.8 M, about 1.6 M, about 1.4 M, about 1.2 M, about 1.0 M, about 0.8 M, about 0.6 M, about 0.4 M, or about 0.2 M.

In certain embodiments, the invention relates to any one of the aforementioned methods, wherein the first flow rate is from about 0.1 to about 10 mL/min. In certain embodiments, the invention relates to any one of the aforementioned methods, wherein the second flow rate is from about 0.1 to about 10 mL/min. In certain embodiments, the invention relates to any one of the aforementioned methods, wherein the first flow rate or the second flow rate is about 0.1 mL/min, about 0.5 mL/min, about 1.0 mL/min, about 1.5 mL/min, about 2.0 mL/min, about 2.5 mL/min, about 3.0 mL/min, about 4.0 mL/min, about 4.5 mL/min, about 5.0 mL/min, about 5.5 mL/min, about 6.0 mL/min, about 6.5 mL/min, about 7.0 mL/min, about 7.5 mL/min, about 8.0 mL/min, about 8.5 mL/min, about 9.0 mL/min, about 9.5 mL/min, or about 10.0 mL/min. In certain embodiments, the invention relates to any one of the aforementioned methods, wherein the first flow rate or the second flow rate is from about 0.5 mL/min to about 5.0 mL/min.

In certain embodiments, the invention relates to any one of the aforementioned methods, further comprising the step of:

contacting a third fluid with the substance adsorbed or absorbed onto the composite material, thereby releasing a portion of the substance from the composite material; wherein the third fluid is a salt solution; and the salt concentration of the third fluid is less than the salt concentration of the second fluid.

In certain embodiments, the invention relates to a method of making a composite material, comprising the steps of:

combining a monomer, a photoinitiator, a cross-linking agent, and a solvent, thereby forming a monomeric mixture;

contacting a support member with the monomeric mixture, thereby forming a modified support member; wherein the support member comprises a plurality of pores extending through the support member, and the average pore diameter of the pores is about 0.1 to about 25 μm;

covering the modified support member with a polymeric sheet, thereby forming a covered support member; and

irradiating the covered support member for a period of time, thereby forming a composite material.

In certain embodiments, the invention relates to any one of the aforementioned methods, further comprising the step of washing the composite material with a second solvent.

In certain embodiments, the invention relates to any one of the aforementioned methods, wherein the monomer comprises acrylamide, N-acryloxysuccinimide, butyl acrylate or methacrylate, N,N-diethylacrylamide, N,N-dimethylacrylamide, 2-(N,N-dimethylamino)ethyl acrylate or methacrylate, N-[3-(N,N-dimethylamino)propyl]methacrylamide, N,N-dimethylacrylamide, n-dodecyl acrylate, n-dodecyl methacrylate, phenyl acrylate or methacrylate, dodecyl methacrylamide, ethyl acrylate or methacrylate, 2-ethylhexyl methacrylate, hydroxypropyl methacrylate, glycidyl acrylate or methacrylate, ethylene glycol phenyl ether acrylate or methacrylate, n-heptyl acrylate or methacrylate, 1-hexadecyl acrylate or methacrylate, methacrylamide, methacrylic anhydride, octadecyl acrylamide, octylacrylamide, octyl acrylate or methacrylate, propyl acrylate or methacrylate, N-iso-propylacrylamide, stearyl acrylate or methacrylate, styrene, alkylated styrene derivatives, 4-vinylpyridine, vinylsulfonic acid, N-vinyl-2-pyrrolidinone (VP), acrylamido-2-methyl-1-propanesulfonic acid, styrenesulfonic acid, alginic acid, (3-acrylamidopropyl)trimethylammonium halide, diallyldimethylammonium halide, 4-vinyl-N-methylpyridinium halide, vinylbenzyl-N-trimethylammonium halide, methacryloxyethyltrimethylammonium halide, or 2-(2-methoxy)ethyl acrylate or methacrylate.

In certain embodiments, the invention relates to any one of the aforementioned methods, wherein the photoinitiator is present in the monomeric mixture in an amount from about 0.4% (w/w) to about 2.5% (w/w) relative to the total weight of monomer.

In certain embodiments, the invention relates to any one of the aforementioned methods, wherein the photoinitiator is present in the monomeric mixture in about 0.6%, about 0.8%, about 1.0%, about 1.2%, or about 1.4% (w/w) relative to the total weight of monomer.

In certain embodiments, the invention relates to any one of the aforementioned methods, wherein the photoinitiator is selected from the group consisting of 1-[4-(2-hydroxyethoxy)-phenyl]-2-hydroxy-2-methyl-1-propane-1-one, 2,2-dimethoxy-2-phenylacetophenone, benzophenone, benzoin and benzoin ethers, dialkoxyacetophenones, hydroxyalkylphenones, and α-hydroxymethyl benzoin sulfonic esters.

In certain embodiments, the invention relates to any one of the aforementioned methods, wherein the solvent is 1,3-butanediol, di(propylene glycol) propyl ether, N,N-dimethylacetamide, di(propylene glycol) methyl ether acetate (DPMA), water, dioxane, dimethylsulfoxide (DMSO), dimethylformamide (DMF), acetone, ethanol, N-methylpyrrolidone (NMP), tetrahydrofuran (THF), ethyl acetate, acetonitrile, toluene, xylenes, hexane, N-methylacetamide, propanol, or methanol.

In certain embodiments, the invention relates to any one of the aforementioned methods, wherein the monomer or the cross-linking agent or both are present in the solvent in about 10% to about 45% (w/w).

In certain embodiments, the invention relates to any one of the aforementioned methods, wherein the monomer or the cross-linking agent or both are present in the solvent in an amount of about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40% (w/w).

In certain embodiments, the invention relates to any one of the aforementioned methods, wherein the cross-linking agent is selected from the group consisting of bisacrylamidoacetic acid, 2,2-bis[4-(2-acryloxyethoxy)phenyl]propane, 2,2-bis(4-methacryloxyphenyl)propane, butanediol diacrylate and dimethacrylate, 1,4-butanediol divinyl ether, 1,4-cyclohexanediol diacrylate and dimethacrylate, 1,10-dodecanediol diacrylate and dimethacrylate, 1,4-diacryloylpiperazine, diallylphthalate, 2,2-dimethylpropanediol diacrylate and dimethacrylate, dipentaerythritol pentaacrylate, dipropylene glycol diacrylate and dimethacrylate, N,N-dodecamethylenebisacrylamide, divinylbenzene, glycerol trimethacrylate, glycerol tris(acryloxypropyl)ether, N,N′-hexamethylenebisacrylamide, N,N′-octamethylenebisacrylamide, 1,5-pentanediol diacrylate and dimethacrylate, 1,3-phenylenediacrylate, poly(ethylene glycol) diacrylate and dimethacrylate, poly(propylene) diacrylate and dimethacrylate, triethylene glycol diacrylate and dimethacrylate, triethylene glycol divinyl ether, tripropylene glycol diacrylate or dimethacrylate, diallyl diglycol carbonate, poly(ethylene glycol) divinyl ether, N,N′-dimethacryloylpiperazine, divinyl glycol, ethylene glycol diacrylate, ethylene glycol dimethacrylate, N,N′-methylenebisacrylamide, 1,1,1-trimethylolethane trimethacrylate, 1,1,1-trimethylolpropane triacrylate, 1,1,1-trimethylolpropane trimethacrylate (TRIM-M), vinyl acrylate, 1,6-hexanediol diacrylate and dimethacrylate, 1,3-butylene glycol diacrylate and dimethacrylate, alkoxylated cyclohexane dimethanol diacrylate, alkoxylated hexanediol diacrylate, alkoxylated neopentyl glycol diacrylate, aromatic dimethacrylate, caprolactone modified neopentylglycol hydroxypivalate diacrylate, cyclohexane dimethanol diacrylate and dimethacrylate, ethoxylated bisphenol diacrylate and dimethacrylate, neopentyl glycol diacrylate and dimethacrylate, ethoxylated trimethylolpropane triacrylate, propoxylated trimethylolpropane triacrylate, propoxylated glyceryl triacrylate, pentaerythritol triacrylate, tris(2-hydroxy ethyl)isocyanurate triacrylate, di-trimethylolpropane tetraacrylate, dipentaerythritol pentaacrylate, ethoxylated pentaerythritol tetraacrylate, pentaacrylate ester, pentaerythritol tetraacrylate, caprolactone modified dipentaerythritol hexaacrylate, N,N′,-methylenebisacrylamide, diethylene glycol diacrylate and dimethacrylate, trimethylolpropane triacrylate, ethylene glycol diacrylate and dimethacrylate, tetra(ethylene glycol) diacrylate, 1,6-hexanediol diacrylate, divinylbenzene, and poly(ethylene glycol) diacrylate.

In certain embodiments, the invention relates to any one of the aforementioned methods, wherein the mol % of cross-linking agent relative to monomer is about 10%, about 11%, about 12%, about 13%, about 14%, about 15%, about 16%, about 17%, about 18%, about 19%, about 20%, about 21%, about 22%, about 23%, about 24%, or about 25%.

In certain embodiments, the invention relates to any one of the aforementioned methods, wherein the covered support member is irradiated at about 350 nm.

In certain embodiments, the invention relates to any one of the aforementioned methods, wherein the period of time is about 1 minute, about 5 minutes, about 10 minutes, about 15 minutes, about 20 minutes, about 30 minutes, about 45 minutes, or about 1 hour.

In certain embodiments, the invention relates to any one of the aforementioned methods, wherein the composite material comprises macropores.

In certain embodiments, the invention relates to any one of the aforementioned methods, wherein the average pore diameter of the macropores is less than the average pore diameter of the pores.

### EXEMPLIFICATION

The following examples are provided to illustrate the invention. It will be understood, however, that the specific details given in each example have been selected for purpose of illustration and are not to be construed as limiting the scope of the invention. Generally, the experiments were conducted under similar conditions unless noted.

### Example 1

### General Procedures

Preparation of Composite Materials

A composite material was prepared from the monomer solutions described below and the support TR0671 B50 (Hollingsworth & Vose) using the photoinitiated polymerization according to the following general procedure. A weighed support member was placed on a poly(ethylene) (PE) sheet and a monomer or polymer solution was applied to the sample. The sample was subsequently covered with another PE sheet and a rubber roller was run over the sandwich to remove excess solution. In situ gel formation in the sample was induced by polymerization initiated by irradiation with the wavelength of 350 nm for a period of 10 minutes. The resulting composite material was thoroughly washed with RO water and placed into 0.1 N hydrochloric acid for 24 h to hydrolyze residual epoxide groups. (Although the epoxide hydrolysis step was executed in each of the following examples, this step may be omitted). Membrane was stored in water for 24 h and then dried at room temperature. To determine the amount of gel formed in the support, the sample was dried in an oven at 50° C. to a constant mass. The mass gain due to gel incorporation was calculated as a ratio of add-on mass of the dry gel to the initial mass of the porous support.

Analysis of Flux and Binding Capacity of Composite Materials

Water flux measurements through the composite materials were carried out after the samples had been washed with water. As a standard procedure, a sample in the form of a disk of diameter 7.8 cm was mounted on a sintered grid of 3-5 mm thickness and assembled into a cell supplied with compressed nitrogen at a controlled pressure. The cell was filled with deionised water and pressure of 100 kPa was applied. The water that passed through the composite material in a specified time was collected in a pre-weighed container and weighed. All experiments were carried out at room temperature and at atmospheric pressure at the permeate outlet. Each measurement was repeated three or more times to achieve reproducibility of ±5%.

Protein adsorption experiments were carried out with lysozyme and hIgG. In an adsorption step, a composite material sample in a form of a single membrane disk of diameter 25 mm was mounted on a sintered grid of 2 mm thickness in a dead-end cell.

The feed solution supplied to the cell by a peristaltic pump (model P-1, Pharmacia Biotech). The permeate outlet was connected to a multi-wavelength UV detector (Waters 490). The detector plotter output was connected to a digital multimeter with PC interface and through the multimeter to PC. The detector output was recorded every minute with an accuracy of ±1 mV.

The cell and the membrane sample were primed by passing 20 mM sodium phosphate buffer containing ammonium sulphate salt of various concentrations at pH 7.0 until a stable base line in the UV detector at 280 nm was established. In the next step, the cell was emptied and refilled with the feed—lysozyme or hIgG solution in corresponding buffers. All buffers, lysozyme and hIgG solutions were filtered through a cellulose acetate membrane filter with pore size of 0.2 μm. The pump was turned on immediately along with the PC recording of the detector output. Permeate samples were collected and weighed to check the flow rate.

### Example 2

### Butyl-Functionalized Composite Material

A 23 wt % solution was prepared by dissolving glycidyl methacrylate (GMA) monomer, butyl methacrylate (BuMe) co-monomer and trimethylolpropane trimethacrylate (TRIM-M) cross-linker in a molar ratio of 1:0.15:0.2, respectively, in a solvent mixture containing 22.4 wt % 1,3-butanediol, 54.3 wt % di(propylene glycol) propyl ether and 23.3 wt % N,N′-dimethylacetamide. The photoinitiator Irgacure® 2959 was added in the amount of 1 wt % with respect to the mass of the monomers.

Several samples similar to that described above were prepared and averaged to estimate the mass gain of the composite material. The substrate gained 180% of the original weight in this treatment.

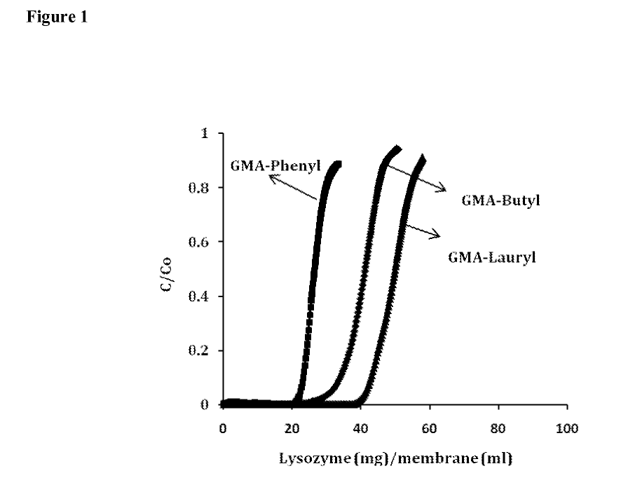

The composite material produced by this method had a water flux in the range of 1,200-1,400 kg/m²h at 100 kPa. The lysozyme (LYS) and hIgG adsorption characteristics of the composite material were examined using the general procedure for a single membrane disk as described above. The concentration of the lysozyme used in this experiment was 0.45 g/L in 20 mM sodium phosphate buffer containing 2.0 M (NH₄)₂SO₄, at pH 7.0 and hIgG—0.5 g/L in 20 mM sodium phosphate buffer containing 1.5 N (NH₄)₂SO₄ at pH 7.0. The flow rate was 10 bed volume (BV)/min. A plot of the concentration of LYS in the permeate (membrane breakthrough) vs. the LYS dynamic binding capacity (mg/mL) is shown in FIG. 1. The composite material had a LYS and hIgG binding capacity of 34 mg/mL_{membrane} and 41 mg/mL_{membrane} correspondingly at 10% breakthrough. Desorption was effected with 20 mM sodium phosphate buffer. The elution fractions were collected for spectrophotometric determinations at 280 nm. The recovery was estimated from the volume and the absorbance of the elution sample. The results indicated a LYS and hIgG recovery of 90% and 75%, respectively.

### Example 3

### Phenyl-Functionalized Composite Material

A 32 wt % solution was prepared by dissolving glycidyl methacrylate (GMA) monomer, phenyl acrylate (PhA) co-monomer and trimethylolpropane trimethacrylate (TRIM-M) cross-linker in a molar ratio of 1:0.1:0.13, respectively, in a solvent mixture containing 22.3 wt % 1,3-butanediol, 55.0 wt % di(propylene glycol) propyl ether and 22.7 wt % N,N′-dimethylacetamide. The photoinitiator Irgacure® 2959 was added in the amount of 1 wt % with respect to the mass of the monomers.

Several samples similar to that described above were prepared and averaged to estimate the mass gain of the composite material. The substrate gained 170% of its original weight in this treatment.

The composite material produced by this method had a water flux in the range of 1,200-1,300 kg/m²h at 100 kPa. The lysozyme (LYS) and hIgG adsorption characteristics of the composite material were examined using the general procedure for a single membrane disk described above (Example 2). The concentration of the protein used in this experiment and flow rates were the same as describe in Example 2. A plot of the concentration of LYS in the permeate vs. the LYS dynamic binding capacity (mg/mL) is shown in FIG. 1. The composite material showed a LYS and hIgG binding capacity of 24 mg/mL_{membrane} and 28.2 mg/mL_{membrane} at 10% breakthrough, respectively. The recovery of LYS was found in the range of 85-90%, and of hIgG was 70%.

### Example 4

### Dodecyl-Functionalized Composite Material

A 25.7 wt % solution was prepared by dissolving glycidyl methacrylate (GMA) monomer, lauryl methacrylate (LMA, dodecyl methacrylate) co-monomer and trimethylolpropane trimethacrylate (TRIM-M) cross-linker in a molar ratio of 1:0.1:0.23, respectively, in a solvent mixture containing 24.3 wt % 1,3-butanediol, 53.6 wt % di(propylene glycol) propyl ether and 22.1 wt % N,N′-dimethylacetamide. The photoinitiator Irgacure® 2959 was added in the amount of 1 wt % with respect to the mass of the monomers.

Several samples similar to that described above were prepared and averaged to estimate the mass gain of the composite material. The substrate gained 180% of its original weight in this treatment.

The composite material produced by this method had a water flux in the range of 1,200-1,300 kg/m²h at 100 kPa. A plot of the concentration of LYS in the permeate vs. the LYS dynamic binding capacity (mg/mL) is shown in FIG. 1. The composite material showed a LYS and hIgG binding capacity of 41 mg/mL_{membrane} and 49.4 mg/mL_{membrane} at 10% breakthrough, respectively. The LYS and hIgG recoveries were 80-85% and 75%, respectively.

### Example 5

### Butyl-Functionalized Composite Materials with Vinyl Pyrrolidinone and Acrylamide Co-Monomers

A 19.0 wt % solution was prepared by dissolving 1-vinyl-2-pyrrolidinone (VP) monomer, acrylamide (AAm) co-monomer-1, butyl methacrylate (BuMe) co-monomer-2 and trimethylolpropane trimethacrylate (TRIM-M) cross-linker in a molar ratio of 1:0.1:0.14:0.27, respectively, in a solvent mixture containing 99 wt % di(propylene glycol) methyl ether acetate (DPMA) and 1 wt % DI water. The photoinitiator Irgacure® 2959 was added in the amount of 1 wt % with respect to the mass of the monomers.

Several samples similar to that described above were prepared and averaged to estimate the mass gain of the composite material. The substrate gained 150% of its original weight in this treatment.

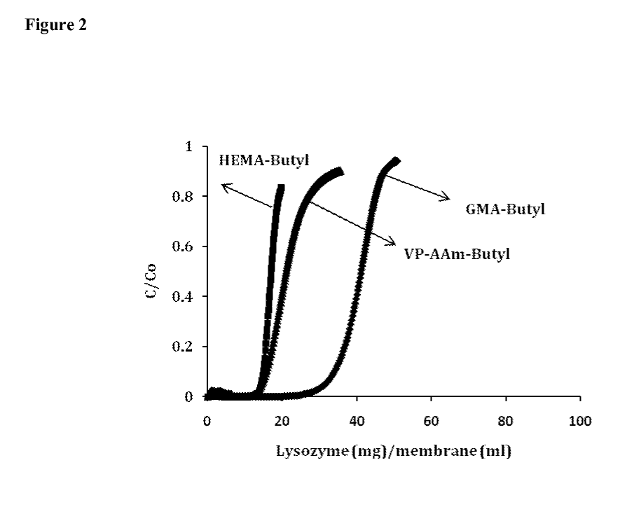

The composite material produced by this method had a water flux in the range of 1,000-1,100 kg/m²h at 100 kPa. The protein (lysozyme (LYS)) and hIgG adsorption characteristics of the composite material were examined using the general procedure for a single membrane disk described above (Example 2). The concentration of lysozyme/hIgG used in this experiment was the same as described in Example 2. The flow rate was 8 bed volume (BV)/min. A plot of the concentration of LYS in the permeate vs. the LYS dynamic binding capacity (mg/mL) is shown in FIG. 2. The composite material showed a LYS and hIgG binding capacity of 15 mg/mL_{membrane} and 20 mg/mL_{membrane} at 10% breakthrough, respectively. The LYS/hIgG recovery was in range of 70-75%.

### Example 6

### Butyl-Functionalized Composite Materials with Hydroxyethyl Methacrylate Co-Monomer

A 36.0 wt % solution was prepared by dissolving 2-hydroxyethyl methacrylate (HEMA) monomer, butyl methacrylate (BuMe) co-monomer and trimethylolpropane trimethacrylate (TRIM-M) cross-linker in a molar ratio of 1:0.1:0.12, respectively, in di(propylene glycol) methyl ether acetate (DPMA). The photoinitiator Irgacure® 2959 was added in the amount of 1 wt % with respect to the mass of the monomers.

A composite material was prepared from the solution and the support TR0671 B50 (Hollingsworth & Vose) using the photoinitiated polymerization according to the general procedure describe above (Example 1). The irradiation time used was 10 minutes at 350 nm. The composite material was removed from between the polyethylene sheets, washed with RO water. The membrane was stored in water for 24 h and dried at room temperature.

Several samples similar to that described above were prepared and averaged to estimate the mass gain of the composite material. The substrate gained 130% of its original weight in this treatment.

The membrane was characterized in terms of water flux and lysozyme/hIgG binding capacity as described in Example 2.

The composite material produced by this method had a water flux in the range of 3,200-3,500 kg/m²h at 100 kPa. The lysozyme/hIgG adsorption characteristic of the composite material was examined using the general procedure. Two membrane disks were used. The concentration of lysozyme/hIgG used in this experiment was the same as described in Example 2. The flow rate was 5 bed volume (BV)/min. A plot of the concentration of LYS in the permeate vs. the LYS dynamic binding capacity (mg/mL) is shown in FIG. 2. The composite material showed a LYS and hIgG binding capacity of 15 mg/mL_{membrane} and 17 mg/mL_{membrane} at 10% breakthrough, respectively. The recovery of LYS was found to be in the range of 80%, and of hIgG was in the range of 70%.

### Example 7

### Gradient Chromatography

To obtain the protein linear-gradient retention data, the poly(AAm-co-VP-co-BuMe) membrane prepared as described in Example 5 (25 mm in diameter; 0.14 mL) was used. Three proteins, varying in molecular weight and in the hydrophobicity of their surfaces, were examined in separation experiment. Proteins used were: myoglobin (from equine skeletal muscle, MW 17 kDa), lysozyme (from chicken egg white, MW 14.3 kDa), and α-chymotrypsinogen A (type II, from bovine pancreas, MW 25.7 kDa) (Sigma-Aldrich). All test salts and proteins, ammonium sulfate, and sodium monobasic and dibasic phosphate were purchased from Sigma-Aldrich. A Waters 600E HPLC system was used for carrying out the membrane chromatographic studies. A 2-mL sample loop was used for injecting protein solutions in separation experiments. The UV absorbance (at 280 nm) of the effluent stream from the Pall membrane holder and the system pressure were continuously recorded. A solution containing 2 M ammonium sulfate (pH 7.0) was chosen as a binding buffer which was prepared using 20 mM sodium phosphate buffer (pH 7.0) as a base buffer. The binding buffer was referred to as buffer A, and the elution buffer—20 mM sodium phosphate (pH 7.0)—as buffer B. Proteins were dissolved in binding buffer (buffer A) to prepare 2 mg/mL solutions. The protein solutions were mixed in a ratio of 3:1:3 (myoglobin:lysozyme:α-chymotrypsinogen A).

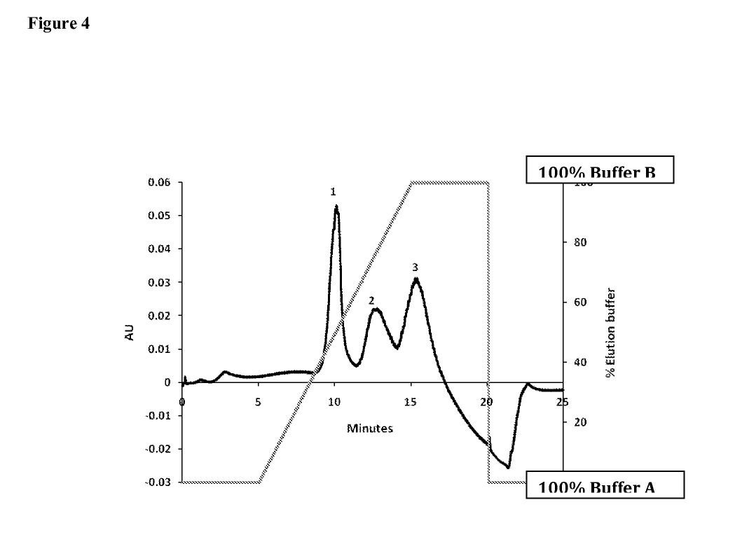

In chromatographic experiments, buffer A was passed through the membrane until a stable UV absorbance baseline was obtained. Then, 150 μL of protein mixture was injected using a 2-mL sample loop. Binding buffer was run for 5 min at 2 mL/min. Subsequently, elution was achieved in 15-min at a 2 mL/min descending salt gradient (0% buffer B—100% buffer B). FIG. 4 shows very good analytical separation of myoglobin, lysozyme and α-chymotrypsinogen using one membrane disk at a flow rate of 2 mL/min. The first peak in FIG. 4 was due to the bound and subsequently eluted myoglobin. The second peak is lysozyme, and the third peak is α-chymotrypsinogen. The peak identities were confirmed in single-protein experiments. The elution times (t_{R}) of these proteins are presented in Table 2.

TABLE 2
Protein retention time based on individual capture/elution experiments

| Protein | Elution time (min) |
|---|---|
| Myoglobin | 10 |
| Lysozyme | 12.5 |
| α-Chymotrypsinogen-A | 15.3 |

### Example 8

### Membrane Performance

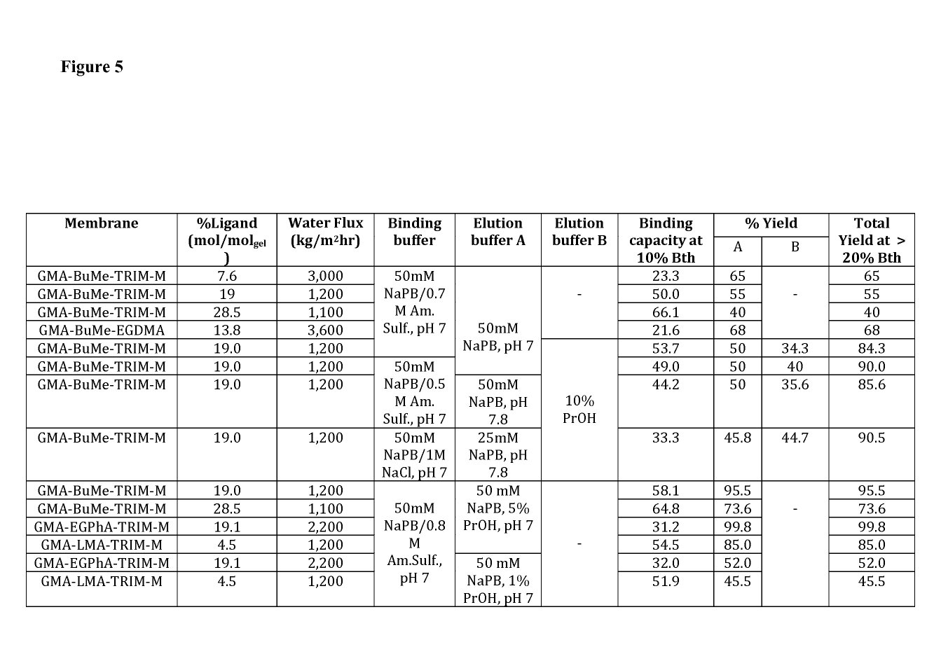

FIG. 5 tabulates a summary of various composite materials of the invention and various performance characteristics. Each of the samples was made, hydrolyzed with 0.1 M HCl, dried in an oven at 50° C., and re-wet for use, an important consideration for a practical material.

### Example 9

### Phenyl-Functionalized Composite Materials

This example illustrates a method of preparing a composite material of the present invention with phenyl functional group.

A 32 wt % solution was prepared by dissolving glycidyl methacrylate (GMA) monomer, ethylene glycol phenyl ether methacrylate (EGPhA) co-monomer and trimethylolpropane trimethacrylate (TRIM-M) cross-linker in a molar ratio of 1:0.2:0.18, respectively, in a solvent mixture containing 23.1 wt % 1,3-butanediol, 54.0 wt % di(propylene glycol)propyl ether and 22.9 wt % N,N′-dimethylacetamide. The photo-initiator Irgacure® 2959 was added in the amount of 1 wt % with respect to the mass of the monomers.

A composite material was prepared from the solution and the support TR0671 B50 (Hollingsworth & Vose) using the photoinitiated polymerization according to the general procedure describe above (Example 1). The irradiation time used was 10 minutes at 350 nm. The composite material was removed from between the polyethylene sheets, washed with RO water and placed into 0.1 N hydrochloric acid for 24 hrs to hydrolyze epoxy groups. Membrane was stored in water for 24 h and then dried at room temperature.

Several samples similar to that described above were prepared and averaged to estimate the mass gain of the composite material. The substrate gained 180% of the original weight in this treatment.

The composite material produced by this method had a water flux of 2,200 kg/m² hr at 100 kPa. The hIgG adsorption characteristics of the composite material were examined using a single layer inserted into a stainless steel Natrix disk holder attached to Waters 600E HPLC system equipment. The concentration of the hIgG used in this experiment was 0.5 g/L in 50 mM sodium phosphate buffer containing 0.8 M (NH₄)₂SO₄, at pH 7.0. The flow rate was 10 bed volume (BV)/min. The composite material showed hIgG binding capacity of 31.2 mg/ml_{membrane} at 10% breakthrough. Recovery for hIgG was 99.8% using elution buffer containing 50 mM sodium phosphate with 5% (w/w) iso-propanol, pH 7.0.

### INCORPORATION BY REFERENCE

All of the U.S. patents and U.S. patent application publications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

We claim:

1. A composite material, comprising:
a support member, comprising a plurality of pores extending through the support member; and
a macroporous cross-linked gel, comprising a plurality of macropores, and a plurality of pendant hydrophobic moieties;
wherein the macroporous cross-linked gel is located in the pores of the support member; and the average pore diameter of the macropores is less than the average pore diameter of the pores.

2. The composite material of claim 1, wherein the macroporous cross-linked gel comprises a polymer derived from acrylamide, N-acryloxysuccinimide, butyl acrylate or methacrylate, N,N-diethylacrylamide, N,N-dimethylacrylamide, 2-(N,N-dimethylamino)ethyl acrylate or methacrylate, 2-(N,N-diethylamino)ethyl acrylate or methacrylate N-[3-(N,N-dimethylamino)propyl]methacrylamide, N,N-dimethylacrylamide, n-dodecyl acrylate, n-dodecyl methacrylate, phenyl acrylate or methacrylate, dodecyl methacrylamide, ethyl acrylate or methacrylate, 2-ethylhexyl acrylate or methacrylate, hydroxypropyl acrylate or methacrylate, glycidyl acrylate or methacrylate, ethylene glycol phenyl ether acrylate or methacrylate, n-heptyl acrylate or methacrylate, 1-hexadecyl acrylate or methacrylate, methacrylamide, methacrylic anhydride, octadecyl acrylamide, octylacrylamide, octyl acrylate or methacrylate, propyl acrylate or methacrylate, N-iso-propylacrylamide, stearyl acrylate or methacrylate, styrene, alkylated styrene derivatives, 4-vinylpyridine, vinylsulfonic acid, N-vinyl-2-pyrrolidinone, acrylamido-2-methyl-1-propanesulfonic acid, styrenesulfonic acid, alginic acid, (3-acrylamidopropyl)trimethylammonium halide, diallyldimethylammonium halide, 4-vinyl-N-methylpyridinium halide, vinylbenzyl-N-trimethylammonium halide, methacryloxyethyltrimethylammonium halide, or 2-(2-methoxy)ethyl acrylate or methacrylate.

3. The composite material of claim 1, wherein the pendant hydrophobic moieties are ethyl, butyl, hexyl, 2-ethylhexyl, dodecyl, stearyl, hydroxypropyl, phenyl, ether, or poly(propylene glycol) groups.

4. The composite material of claim 1, wherein the macroporous cross-linked gel comprises a polymer derived from a monomer with a log P value (octanol-water) from about 1 to about 7.

5. The composite material of claim 1, wherein the macroporous cross-linked gel comprises a polymer derived from a first monomer and a second monomer, the first monomer has a log P value (octanol-water) from about 1 to about 7; and the second monomer has a log P value (octanol-water) from about −1 to about 1.

6. The composite material of claim 5, wherein the molar ratio of the first monomer to the second monomer is about 0.01:1 to about 1:1.

7. The composite material of claim 1, wherein the macroporous cross-linked gel comprises macropores; the macroporous cross-linked gel has a volume porosity from about 30% to about 80%; and the macropores have an average pore diameter from about 10 nm to about 3000 nm.

8. The composite material of claim 7, wherein the average pore diameter of the macropores is about 25 nm to about 1500 nm.

9. The composite material of claim 1, wherein the composite material is a membrane.

10. The composite material of claim 1, wherein the support member has a void volume; and the void volume of the support member is substantially filled with the macroporous cross-linked gel.

11. The composite material of claim 1, wherein the support member comprises a polymer; the support member is about 10 μm to about 500 μm thick; the pores of the support member have an average pore diameter from about 0.1 μm to about 25 μm; and the support member has a volume porosity from about 40% to about 90%.

12. The composite material of claim 1, wherein the support member comprises a polyolefin.

13. The composite material of claim 1, wherein the support member comprises a polymeric material selected from the group consisting of polysulfones, polyethersulfones, polyphenyleneoxides, polycarbonates, polyesters, cellulose and cellulose derivatives.

14. The composite material of claim 1, wherein the support member comprises a fibrous woven or non-woven fabric comprising a polymer; the support member is from about 10 μm to about 2000 μm thick; the pores of the support member have an average pore diameter of from about 0.1 μm to about 25 μm; and the support member has a volume porosity from about 40% to about 90%.

15. A method, comprising the step of:
contacting at a first flow rate a first fluid comprising a substance with a composite material of claim 1, thereby adsorbing or absorbing a portion of the substance onto the composite material.

16. The method of claim 15, wherein the fluid flow path of the first fluid is substantially perpendicular to the pores of the support member.

17. The method of claim 15, wherein the fluid flow path of the first fluid is substantially through the macropores of the composite material.

18. The method of claim 15, further comprising the step of:
contacting at a second flow rate a second fluid with the substance adsorbed or absorbed onto the composite material, thereby releasing a portion of the substance from the composite material.

19. The method of claim 18, wherein the fluid flow path of the second fluid is substantially perpendicular to the pores of the support member.

20. The method of claim 18, wherein the fluid flow path of the second fluid is substantially through the macropores of the composite material.

21. The method of claim 15, wherein the macroporous gel displays a specific interaction for the substance; and the specific interaction is a hydrophobic interaction.

22. The method of claim 15, wherein the substance is a biological molecule or biological ion.

23. The method of claim 22, wherein the biological molecule or biological ion is selected from the group consisting of albumins, lysozyme, viruses, cells, γ-globulins of human and animal origins, immunoglobulins of human and animal origins, proteins of recombinant and natural origins, polypeptides of synthetic and natural origins, interleukin-2 and its receptor, enzymes, monoclonal antibodies, trypsin and its inhibitor, cytochrome C, myoglobin, myoglobulin, α-chymotrypsinogen, recombinant human interleukin, recombinant fusion protein, nucleic acid derived products, DNA of synthetic and natural origins, and RNA of synthetic and natural origins.

24. A method of making a composite material, comprising the steps of:
combining a monomer, a photoinitiator, a cross-linking agent, and a solvent, thereby forming a monomeric mixture;
contacting a support member with the monomeric mixture, thereby forming a modified support member; wherein the support member comprises a plurality of pores extending through the support member, and the average pore diameter of the pores is about 0.1 to about 25 μm;
covering the modified support member with a polymeric sheet, thereby forming a covered support member; and
irradiating the covered support member for a period of time, thereby forming a composite material.
